(19) United States
(12) Patent Application Publication
(10) Pub. No.: US 2008/0175808 A1
(43) Pub. Date: Jul. 24, 2008

(54) KERATIN-ENHANCING COMPOSITIONS

(75) Inventor: Florentina PAVEL, Hillsborough, NJ (US)

Correspondence Address: OBLON, SPIVAK, MCCLELLAND MAIER & NEUSTADT, P.C., 1940 DUKE STREET, ALEXANDRIA, VA, 22314, US

(73) Assignee: L'Oreal S.A., Paris (FR)

(21) Appl. No.: 11/972,102

(22) Filed: Jan. 10, 2008

Related U.S. Application Data
(60) Provisional application No. 60/884,758, filed on Jan. 12, 2007.

(51) Int. Cl. A61K 8/37; A61K 8/90; A61Q 1/10

(52) U.S. Cl. 424/70.7

(57) ABSTRACT

The invention relates to compositions for keratin materials (hair or eyelashes) such as mascaras, topcoats and basecoats containing at least one tackifier and at least one semi-crystalline alkyl(meth)acrylate.

### CROSS-REFERENCE TO PRIOR APPLICATIONS

This application claims priority to U.S. provisional application 60/884,758 filed Jan. 12, 2007, the entirety of which is hereby incorporated by reference.

### FIELD OF THE INVENTION

The present invention generally relates to compositions for keratin materials (hair or eyelashes) such as, for example, mascaras, topcoats and basecoats comprising at least one tackifier, at least one block copolymer and at least one semi-crystalline alkyl(meth)acrylate. Such compositions, when applied to keratin materials, impart improved length to the keratin materials. Such compositions can also possess improved properties and characteristics such as, for example, increased volume of the keratin material, improved transfer-resistance of the composition, improved waterproofing characteristics and/or improved long-wear properties.

### BACKGROUND OF THE INVENTION

Many mascaras and other cosmetic compositions have been developed for improved wear, transfer-resistance, and length/volume properties. Such properties are typically accomplished by the use of ingredients such as film forming agents and/or tackifying agents. Some mascaras, upon application, form “faux fibers” on the eyelashes owing to the presence of such ingredients: that is, they form extensions on the eyelashes (in situ) which provide the eyelashes with the appearance of increased length. Unfortunately, such faux fibers are brittle and break or collapse easily, thereby negating any perceived lengthening associated with such compositions. Thus, there remains a need in the art for improved cosmetic compositions, particularly mascaras, which possess significantly improved lengthening properties, particularly lengthening properties associated with the formation and/or maintenance of faux fibers.

Accordingly, one aspect of the present invention is a makeup, care and/or treatment composition for keratin materials such as hair or eyelashes which is able to provide significantly improved cosmetic properties to the keratin materials such as those discussed above.

### SUMMARY OF THE INVENTION

The present invention relates to compositions for keratin materials (hair or eyelashes) such as mascaras, topcoats and basecoats comprising at least one tackifier, at least one block copolymer and at least one semi-crystalline alkyl(meth)acrylate.

The present invention also relates to methods of increasing the volume and/or length of keratin materials (hair or eyelashes) comprising applying to the keratin material a keratin material volume- and/or length-increasing effective amount of a composition comprising at least one tackifier, at least one block copolymer and at least one semi-crystalline alkyl(meth)acrylate.

The present invention further relates to methods of making-up keratin materials (hair or eyelashes) comprising applying a keratin material making-up effective amount of a composition comprising at least one tackifier, at least one block copolymer and at least one semi-crystalline alkyl(meth)acrylate to keratin materials in need of such making-up.

The present invention also relates to methods of treating or caring for keratin materials (hair or eyelashes) by applying compositions of the present invention to the keratin materials in an amount sufficient to treat and/or care for the keratin materials.

The present invention further relates to methods of enhancing the appearance of keratin materials (hair or eyelashes) by applying compositions of the present invention to the keratin materials in an amount sufficient to enhance the appearance of the keratin materials.

The present invention also relates to methods of supporting, strengthening, stiffening and/or reinforcing faux fibers by adding a supporting-, strengthening-, stiffening and/or reinforcing-effective amount of at least one semi-crystalline alkyl(meth)acrylate to a composition comprising at least one tackifier and at least one block copolymer which, upon application to a keratin material, forms faux fibers on the keratin material.

It is to be understood that both the foregoing general description and the following detailed description are exemplary and explanatory only, and are not restrictive of the invention.

### DETAILED DESCRIPTION OF THE INVENTION

As used herein, the expression “at least one” means one or more and thus includes individual components as well as mixtures/combinations.

“Transfer resistance” as used herein refers to the quality exhibited by compositions that are not readily removed by contact with another material, such as, for example, a glass, an item of clothing or the skin, for example, when eating or drinking. Transfer resistance may be evaluated by any method known in the art for evaluating such. For example, transfer resistance of a composition may be evaluated by a modified “kiss” test. The modified “kiss” test may involve application of the composition to human eyelashes followed by “kissing” or rubbing a material with the eyelashes, for example, a sheet of paper, after expiration of a certain amount of time following application, such as 2 minutes after application. Similarly, transfer resistance of a composition may be evaluated by the amount of product transferred from a wearer to any other substrate, such as transfer from the eyelashes of an individual to clothing after the expiration of a certain amount of time following application. The amount of composition transferred to the substrate (e.g., clothing or paper) may then be evaluated and compared. For example, a composition may be transfer resistant if a majority of the product is left on the eyelashes. Further, the amount transferred may be compared with that transferred by other compositions, such as commercially available compositions.

“Long wear” compositions as used herein, refers to compositions where at least one property chosen from consistency, texture, and color remains the same as at the time of application, as viewed by the naked eye, after an extended period of time, such as, for example, 1 hour, 2 hours, and further such as 8 hours. Long wear properties may be evaluated by any method known in the art for evaluating such properties. For example, long wear may be evaluated by a test involving the application of a composition to eyelashes and evaluating the consistency, texture and color of the composition after an extended period of time. For example, the consistency, texture and color of a mascara composition may be evaluated immediately following application and these characteristics may then be re-evaluated and compared after an individual has worn the mascara composition for a certain amount of time. Further, these characteristics may be evaluated with respect to other compositions, such as commercially available compositions.

“Waterproof” as used herein refers to the ability to repel water and permanence with respect to water. Waterproof properties may be evaluated by any method known in the art for evaluating such properties. For example, a mascara composition may be applied to false eyelashes, which may then be placed in water for a certain amount of time, such as, for example, 20 minutes. Upon expiration of the pre-ascertained amount of time, the false eyelashes may be removed from the water and passed over a material, such as, for example, a sheet of paper. The extent of residue left on the material may then be evaluated and compared with other compositions, such as, for example, commercially available compositions. Similarly, for example, a composition may be applied to skin, and the skin may be submerged in water for a certain amount of time. The amount of composition remaining on the skin after the pre-ascertained amount of time may then be evaluated and compared. For example, a composition may be waterproof if a majority of the product is left on the wearer, e.g., eyelashes.

“Tackiness” as used herein refers to the adhesion between two substances. For example, the more tackiness there is between two substances, the more adhesion there is between the substances. To quantify “tackiness,” it is useful to determine the “work of adhesion” as defined by IUPAC associated with the two substances. Generally speaking, the work of adhesion measures the amount of work necessary to separate two substances. Thus, the greater the work of adhesion associated with two substances, the greater the adhesion there is between the substances, meaning the greater the tackiness is between the two substances.

Work of adhesion and, thus, tackiness, can be quantified using acceptable techniques and methods generally used to measure adhesion, and is typically reported in units of force time (for example, gram seconds (“g s”)). For example, the TA-XT2 from Stable Micro Systems, Ltd. can be used to determine adhesion following the procedures set forth in the TA-XT2 Application Study (ref: MATI/PO.25), revised January 2000, the entire contents of which are hereby incorporated by reference. According to this method, desirable values for work of adhesion for substantially non-tacky substances include less than about 0.5 g s, less than about 0.4 g s, less than about 0.3 g s and less than about 0.2 g s. As known in the art, other similar methods can be used on other similar analytical devices to determine adhesion.

The cosmetic compositions and methods of the present invention can comprise, consist of, or consist essentially of the essential elements and limitations of the invention described herein, as well as any additional or optional ingredients, components, or limitations described herein or any otherwise useful ingredient found in personal care compositions intended for application to keratin materials.

The composition of the present invention may be in any form suitable for use on eyelashes such as, for example, non-solid anhydrous, oil-free or emulsion compositions (for example, water-in-oil emulsion, oil-in-water emulsion, multiple emulsion (W/O/W or O/W/O), nanoemulsions, etc.). The compositions of the present invention can be mascaras. Generally speaking, mascaras contain colorants such as pigments. Additionally, the compositions of the present invention can be clear or transparent: that is, they can contain little or no colorants. The compositions of the present invention, particularly those with little or no colorants, can be used as a basecoat and/or topcoat for application beneath and/or onto other products applied to eyelashes.

As defined herein, stability is tested by placing the composition in a controlled environment chamber for 8 weeks at 25° C. In this test, the physical condition of the sample is inspected as it is placed in the chamber. The sample is then inspected again at 24 hours, 3 days, 1 week, 2 weeks, 4 weeks and 8 weeks. At each inspection, the sample is examined for abnormalities in the composition such as phase separation if the composition is in the form of an emulsion. The stability is further tested by repeating the 8-week test at 40° C., 37° C., 45° C., 50° C. and/or under freeze-thaw conditions. A composition is considered to lack stability if in any of these tests an abnormality that impedes functioning of the composition is observed. The skilled artisan will readily recognize an abnormality that impedes functioning of a composition based on the intended application.

Semi-Crystalline Alkyl(meth)acrylate

According to the present invention, compositions, particularly compositions for keratin materials such as eyelashes or hair, comprising at least one semi-crystalline alkyl(meth)acrylate are provided. Preferably, the semi-crystalline alkyl(meth)acrylates are non-crosslinked. The term “semi-crystalline” is understood to mean, within the meaning of the invention, compounds, preferably polymers, comprising a crystallizable part and an amorphous part, and exhibiting a first-order reversible phase change temperature, such as a melting temperature (solid-liquid transition).

Suitable examples of semi-crystalline alkyl(meth)acrylates include, but are not limited to, the Intelimer® or Doresco® products from the company Landec, such as those described in the brochure “Intelimer® Polymers” and/or are disclosed in U.S. patent application publication nos. 2006/0292095 and 2006/0263438, the disclosure of both of which is hereby incorporated by reference in their entirety. Specific examples include:

Doresco/Intelimer IPA 13-1®: polystearyl acrylate, m.p. of 49° C. and MW of 145 000; and

Doresco/Intelimer IPA 13-6®: polybehenyl acrylate, m.p. of 66° C.

In accordance with the present invention, it is also possible to use the semi-crystalline polymers obtained by copolymerization of stearyl acrylate and of acrylic acid or of NVP, or by copolymerization of behenyl acrylate and of acrylic acid or NVP, as described in document U.S. Pat. No. 5,519,063 or EP-A-0 550 745, the entire contents of both of which are hereby incorporated by reference.

Preferably, the amount of semi-crystalline alkyl(meth)acrylate(s) present in the composition is an amount sufficient to support, strengthen, stiffen and/or reinforce faux fibers formed upon application of the composition to a keratin material. Prefeably, the amount of semi-crystalline alkyl(meth)acrylate(s) present ranges from about 1% to 30% by weight with respect to the total weight of the composition, more preferably from about 2% to about 25%, more preferably from about 3% to about 20%, and most preferably from about 4% to about 15% by weight with respect to the total weight of the composition, including all ranges and subranges therebetween

Tackifiers

According to the present invention, compositions comprising at least one tackifier are provided. In general, tackifiers can be divided into four different families in terms of their chemistry: hydrocarbon resins, terpenes, amorphous (i.e. non-crystalline) rosins, rosin esters and their derivatives, and pure monomer resins.

Examples of suitable tackifiers, include, but are not limited to, aliphatic hydrocarbon resins, aromatic modified aliphatic hydrocarbon resins, hydrogenated polycyclopentadiene resins, polycyclopentadiene resins, gum rosins, gum rosin esters, wood rosins, wood rosin esters, tall oil rosins, tall oil rosin esters, polyterpenes, aromatic modified polyterpenes, terpene phenolics, aromatic modified hydrogenated polycyclopentadiene resins, hydrogenated aliphatic resin, hydrogenated aliphatic aromatic resins, hydrogenated terpenes and modified terpenes, hydrogenated rosin acids, hydrogenated rosin esters, polyisoprene, partially or fully hydrogenated polyisoprene, polybutenediene, partially or fully hydrogenated polybutenediene, and the like. As is evidenced by some of the cited examples, the tackifier may be fully or partially hydrogenated. The tackifier may also be non-polar, where “non-polar” means that the tackifier is substantially free of monomers having polar groups. Preferably, polar groups are not present; however, if they are present, they are preferably present in an amount of up to about 5% by weight, preferably up to about 2% by weight, and more preferably up to about 0.5% by weight.

In preferred embodiments, the tackifier may have a softening point (Ring and Ball, as measured by ASTM E-28) of about 80° C. to about 150° C., preferably about 100° C. to about 130° C. In other preferred embodiments, the tackifier may be liquid and have an R and B softening point of between about −70° C. and about 70° C.

According to particularly preferred embodiments, the tackifiers are hydrogenated hydrocarbon resins such as a hydrogenated styrene/methyl styrene/indene copolymer e.g., styrene/methyl styrene/indene copolymers which include R1090, R1100, R7100, S1100, and S5100, all which are commercially available from Eastman Chemical under the trade name Regalite®. In other embodiments, aliphatic or aromatic hydrocarbon-based tackifying resins, for instance the resins sold under the name “Piccotac” and “Hercotac” from Hercules or “Escorez” from Exxon, may also be used. It is also to be understood that mixtures of tackifiers may also be employed without departing from the spirit of the invention.

A particularly preferred tackifier for use in the present invention is a hydrogenated hydrocarbon resin such as, for example, a hydrogenated styrene/methyl styrene/indene copolymer, commercially available from Eastman under the tradename Regalite® R1100.

Preferably, the amount of tackifier(s) present in the composition is an amount sufficient to form faux fiber(s) upon application of the composition to a keratin material. Preferably, the tackifier(s) are present in an amount of from about 0.1 to about 30 percent by weight, more preferably from 1 to 20 percent by weight, more preferably from 1 to 10 percent by weight and most preferably from 1 to 5 percent by weight of the total weight of the composition, including all ranges and subranges therebetween

Block Copolymer

According to the preferred embodiments of the present invention, compositions comprising at least one block copolymer are provided. The block copolymers of the present invention are characterized by the presence of at least one “hard” segment, and at least one “soft” segment. Aside from their compositional nature, the hard and soft segments of the block copolymers of the present invention are defined in terms of their respective glass transition temperatures, “T_{g}”. More particularly, the hard segment has a T_{g} of about 50° C. or more, whereas the soft segment has a T_{g} of about 20° C. or less. The glass transition temperature T_{g} for the hard block can range from about 50° C. to about 150° C.; about 60° C. to about 125° C.; about 70° C. to about 120° C.; or about 80° C. to about 110° C. The glass transition temperature T_{g} for the soft segment of the block copolymer can range from about 20° C. to about −150° C.; about 0° C. to about −135° C.; about −10° C. to about −125° C.; and about −25° C. to about −100° C. A more in depth explanation can be found in U.S. Pat. Nos. 5,294,438 and 6,403,070, the entire contents of which are hereby incorporated by reference.

One type of block copolymer which may be employed in the compositions of the present invention is a thermoplastic elastomer. The hard segments of the thermoplastic elastomer typically comprise vinyl monomers in varying amounts. Examples of suitable vinyl monomers include, but are not limited to, styrene, methacrylate, acrylate, vinyl ester, vinyl ether, vinyl acetate, and the like.

The soft segments of the thermoplastic elastomer typically comprise olefin polymers and/or copolymers which may be saturated, unsaturated, or combinations thereof. Suitable olefin copolymers may include, but are not limited to, ethylene/propylene copolymers, ethylene/butylene copolymers, propylene/butylene copolymers, polybutylene, polyisoprene, polymers of hydrogenated butanes and isoprenes, and mixtures thereof.

Thermoplastic elastomers useful in the present invention include block copolymers e.g., di-block, tri-block, multi-block, radial and star block copolymers, and mixtures and blends thereof. A di-block thermoplastic elastomer is usually defined as an A-B type or a hard segment (A) followed by a soft segment (B) in sequence. A tri-block is usually defined as an A-B-A type copolymer or a ratio of one hard, one soft, and one hard segment. Multi-block or radial block or star block thermoplastic elastomers usually contain any combination of hard and soft segments, provided that the elastomers possess both hard and soft characteristics.

In preferred embodiments, the thermoplastic elastomer of the present invention may be chosen from the class of Kraton™ rubbers (Shell Chemical Company) or from similar thermoplastic elastomers. Kraton™ rubbers are thermoplastic elastomers in which the polymer chains comprise a di-block, tri-block, multi-block or radial or star block configuration or numerous mixtures thereof. The Kraton™ tri-block rubbers have polystyrene (hard) segments on each end of a rubber (soft) segment, while the Kraton™ di-block rubbers have a polystyrene (hard) segment attached to a rubber (soft) segment. The Kraton™ radial or star configuration may be a four-point or other multipoint star made of rubber with a polystyrene segment attached to each end of a rubber segment. The configuration of each of the Kraton™ rubbers forms separate polystyrene and rubber domains.

Each molecule of Kraton™ rubber is said to comprise block segments of styrene monomer units and rubber monomer and/or co-monomer units. The most common structure for the Kraton™ triblock copolymer is the linear A-B-A block type styrene-butadiene-styrene, styrene-isoprene-styrene, styrene-ethylenepropylene-styrene, or styrene-ethylenebutylene-styrene. The Kraton™ di-block is preferably the AB block type such as styrene-ethylenepropylene, styrene-ethylenebutylene, styrene-butadiene, or styrene-isoprene. The Kraton™ rubber configuration is well known in the art and any block copolymer elastomer with a similar configuration is within the practice of the invention. Other block copolymers are sold under the tradename Septon (which represent elastomers known as SEEPS, sold by Kurary, Co., Ltd) and those sold by Exxon Dow under the tradename Vector™.

Other thermoplastic elastomers useful in the present invention include those block copolymer elastomers comprising a styrene-butylene/ethylene-styrene copolymer (tri-block), an ethylene/propylene-styrene copolymer (radial or star block) or a mixture or blend of the two. (Some manufacturers refer to block copolymers as hydrogenated block copolymers, e.g. hydrogenated styrene-butylene/ethylene-styrene copolymer (tri-block)).

The amounts of the block (co)polymer or (co)polymers, as well as their structure (di-block, tri-block, etc.), affect the nature of the thermoplastic elastomer, including its gelled form, which may range from fragile to soft/flexible to firm. For instance, soft gels contain relatively high amounts of soft segments, and firm gels contain relatively high amounts of hard segments. The overall properties of the composition may also be affected by including more than one such block copolymer e.g., including a mixture of copolymers. For example, the presence of tri-block copolymers enhances the integrity of the film formed. The gel may also be transparent, translucent or opaque, depending upon the other cosmetically acceptable ingredients added, as described herein.

It is preferred that the styrene content of the block copolymer be less than 30% by weight, preferably less than 25% by weight, and more preferably less than 20% by weight, based on the weight of the block copolymer. This is because of the tendency of block copolymers having a styrene content of greater than 30% by weight to harden/gel in conventional carrier systems. However, in the event that a block copolymer having a styrene content of greater than 30% by weight is used, it may be necessary to also employ a co-solvent or functional ingredient capable of dissolving a styrene block in an amount effective to control the hardening/gelling of the styrene-containing elastomer in the cosmetic composition.

A particularly preferred block copolymer for use in the present invention is a combination of di-block and tri-block copolymers of styrene-ethylene/butylene-styrene, commercially available from Shell Chemical Company under trade name Kraton G1657M. It should be noted, however, that any thermoplastic elastomer of the block copolymer type having at least one soft and at least one hard segment may be used without departing from the spirit of the invention.

According to preferred embodiments, the tackifiers are characterized by their compatibility with at least one segment of the block copolymer. By the term “compatible”, it is meant, for example, that when the block copolymer and tackifier are mixed, the combination of at least one segment of the block copolymer with the tackifier forms a polymer blend having a single glass transition temperature T_{g} which may be measured by DMA, DSC or neutron light scattering.

The compatibility of the block copolymer and the tackifier may also be defined in terms of solubility parameters. The solubility parameter δ according to the Hansen solubility space is defined in the article “Solubility Parameter Values” by Eric A. Grulke in the work “Polymer Handbook” 3^{rd} edition, Chapter VII, pages 519-559, the entire content of which is hereby incorporated by reference, by the relationship:

δ=(d_{D}²+d_{P}²+d_{H}²)^{1/2}, in which:

d_{D} characterizes the London dispersion forces resulting from the formation of dipoles induced during molecular impacts,

d_{P} characterizes the forces of Debye interactions between permanent dipoles,

d_{H} characterizes the forces of specific interactions (hydrogen bond, acid/base or donor/acceptor type and the like). The definition of the solvents in the three-dimensional solubility space according to Hansen is given in the article by C. M. Hansen: “The three-dimensional solubility parameters” J. Paint Technol., 39, 105(1967), the entire content of which is hereby incorporated by reference.

The at least one tackifier used in the present invention preferably has a solubility parameter corresponding to δ and the block copolymer preferably has at least one segment whose solubility parameter corresponds to δ±2, preferably δ±1.7, more preferably δ±1.5, more preferably δ±1.3, more preferably δ±1.0, more preferably δ±0.7, more preferably δ±0.5, and more preferably δ±0.3.

The block copolymer will preferably have a solubility parameter, relative to the tackifier component, of δ±2, more preferably δ±1.7, more preferably δ±1.5, more preferably δ±1.3, more preferably δ±1.0, more preferably δ±0.7, more preferably δ±0.5, and more preferably δ±0.3.

In the composition of the present invention, the block copolymer(s) are preferably present in an amount of from about 1 to about 20 percent by weight, more preferably from 1 to 15 percent by weight, more preferably from 1 to 10 percent by weight and most preferably from 2 to 8 percent by weight of the total weight of the composition, including all ranges and subranges therebetween.

According to particularly preferred embodiments, the compositions of the present invention comprise an equivalent amount of the at least one block copolymer and the at least one tackifier, or more of the at least one block copolymer as compared to the at least one tackifier. For example, the at least one block copolymer and the at least one tackifier can be present in ratios of 1.25, 1.00, 0.75, 0.50, 0.33, 0.25, and 0.10, including all ranges and subranges therebetween, with ratios from 1.00 to 0.33 being particularly preferred.

Solvents

According to preferred embodiments, the compositions of the present invention further comprise at least one solvent capable of solubilizing the hard or soft segment of the block copolymer. Such suitable solvents are typically characterized in terms of their viscosity at room temperature, weight average molecular weight and/or solubility parameter in relation to the at least one hard segment of the block copolymer.

Solvent(s) capable of solubilizing the hard segment of the block copolymer will preferably have a viscosity, at room temperature, of from about 1 to about 200 cps, more preferably from 1 to 150 cps, more preferably from 1 to 100 cps, more preferably from 2 to 60 cps, and more preferably from 2 to 40 cps.

Solvent(s) capable of solubilizing the hard segment of the block copolymer used in the present invention will preferably have a solubility parameter corresponding to δ′ and the block copolymer will preferably have at least one hard segment whose solubility parameter corresponds to δ′±2, more preferably δ′±1.7, more preferably δ′±1.5, more preferably δ′±1.3, more preferably δ′±1.0, more preferably δ′±0.7, more preferably δ′±0.5, and more preferably δ′±0.3.

Examples of nonvolatile solvents capable of solubilizing the hard segment of the block copolymer which can be used in the invention include, but are not limited to, monoesters, diesters, triesters, mixed aliphatic and/or aromatic, polar oils such as: hydrocarbon-based oils of animal origin, such as perhydrosqualene; hydrocarbon-based plant oils such as liquid triglycerides of fatty acids and of glycerol, in which the fatty acids may have varied chain lengths, these chains being linear or branched, and saturated or unsaturated; these oils can be chosen, for example, from wheat germ oil, sunflower oil, corn oil, soybean oil, marrow oil, grapeseed oil, blackcurrant seed oil, sesame oil, hazelnut oil, apricot oil, macadamia oil, castor oil, avocado oil, karite butter, sweet almond oil, cotton oil, alfalfa oil, poppy oil, pumpkin oil, evening primrose oil, millet oil, barley oil, quinoa oil, olive oil, rye oil, safflower oil, candlenut oil, passion flower oil, musk rose oil and caprylic/capric acid triglycerides such as those sold by the company Stéarineries Dubois or those sold under the names Miglyol 810, 812 and 818 by the company Dynamit Nobel; natural or synthetic esters of formula R₁COOR₂, wherein R₁ is a higher fatty acid residue comprising 7 to 19 carbon atoms, and R₂ is a branched hydrocarbon-based chain comprising 3 to 20 carbon atoms, such as, for example, purcellin oil (cetostearyl octanoate), isopropyl myristate and alkyl or polyalkyl octanoates, decanoates or ricinoleates; synthetic ethers of formula R³COR⁴, wherein R³ is a C₃ to C₁₉ alkyl radical, and R⁴ is a C₃ to C₂₀ alkyl radical; fatty alcohols comprising at least 12 carbon atoms, such as octyldodecanol or oleyl alcohol; cyclic hydrocarbons such as (alkyl)cycloalkanes, wherein the alkyl chain is linear or branched, saturated or unsaturated and comprises 1 to 30 carbon atoms, such as cyclohexane or dioctylcyclohexane; aromatic hydrocarbons, for example, alkenes such as benzene, toluene, 2,4-dimethyl-3-cyclohexene, dipentene, p-cymene, naphthalene or anthracene, and esters such as isostearyl benzoate; primary, secondary or tertiary amines such as triethanolamine; and mixtures thereof. In one embodiment, synthetic esters such as isopropyl myristate are used.

Preferred esters are those having a weight average molecular weight (Mw) in the range of about 100 to about 600, preferably from 100 to 500. Examples thereof include, but are not limited to, C12-15 alkyl benzoate, isopropyl myristate (Mw=270), isopropyl palmitate (Mw=300), isononyl isononanoate, cetyl ethylhexanoate (Mw=368), neopentyl glycol diethylhexanoate (Mw=356), diisopropyl sebacate (Mw=286).

Solvent(s) capable of solubilizing the hard segment of the block copolymer, if present, may typically be present in the composition of the invention in an amount of up to about 85% by weight; up to 75% by weight; up to 55% by weight; up to 45% by weight; up to 40% by weight; up to 30% by weight; up to 20% by weight; up to 10% by weight; and up to 5% by weight, based on the weight of the composition.

Solvents capable of solubilizing the soft segment of the block copolymer which may be used in accordance with preferred embodiments of the present invention are typically characterized in terms of their viscosity at room temperature, weight average molecular weight and/or solubility parameter in relation to the at least one soft segment of the block copolymer.

Solvent(s) capable of solubilizing the soft segment of the block copolymer will preferably have a viscosity, at room temperature, of from about 1 to about 50 cps, more preferably from 1 to 40 cps, more preferably from 1 to 30 cps, more preferably from 2 to 20 cps, and more preferably from 2 to 10 cps.

Solvent(s) capable of solubilizing the soft segment of the block copolymer used in the present invention will preferably have a solubility parameter corresponding to δ′ and the block copolymer will preferably have at least one soft segment whose solubility parameter corresponds to δ′±2, more preferably δ′±1.7, more preferably δ′±1.5, more preferably δ′±1.3, more preferably δ′±1.0, more preferably δ′±0.7, more preferably δ′±0.5, and more preferably δ′±0.3.

Solvent(s) capable of solubilizing the soft segment of the block copolymer may be selected from volatile solvents and nonvolatile solvents. The expression “volatile solvent” means a solvent that is capable of evaporating at room temperature from a support onto which it has been applied, in other words a solvent which has a measurable vapor pressure at room temperature. See, U.S. Pat. No. 6,656,458, the entire content of which is hereby incorporated by reference.

Representative examples of suitable volatile organic solvents include, but are not limited to, volatile hydrocarbon-based oils. The expression “hydrocarbon-based oil” means oil containing only hydrogen and carbon atoms. Examples of volatile hydrocarbon-based oils include isoparaffins, i.e., branched alkanes containing from 8 to 16 carbon atoms, and in particular isododecane (also known as 2,2,4,4,6-pentamethylheptane). It is also possible to use mixtures of such isoparaffins. Other volatile hydrocarbon-based oils, such as petroleum distillates, can also be used.

Representative examples of suitable volatile silicone solvents include, but are not limited to, linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Suitable nonvolatile solvents which can be used are those having a weight average molecular weight in the range of about 150 to about 450, preferably from 200 to 350. Examples thereof include, but are not limited to, hydrogenated polydecene, hydrogenated polyisobutene, isoeicosane, polydecene and polybutene.

Solvent(s) capable of solubilizing the soft segment of the block copolymer, if present, may typically be present in the composition of the invention in an amount of up to about 85% by weight; up to 75% by weight; up to 55% by weight; up to 45% by weight; up to 40% by weight; up to 30% by weight; up to 20% by weight; up to 10% by weight; and up to 5% by weight, based on the weight of the composition.

According to preferred embodiments of the present invention, at least one co-solvent having a high molecular weight and high viscosity may also be used in the invention compositions.

Examples of suitable high viscosity co-solvents which are compatible with the hard segment of the block copolymer include, but are not limited to, capric/caprylic triglyceride (Mw=500), diisopropyl dimer dilinoleate (Mw=644), diisostearyl fumarate (Mw=620), diisostearyl malate (Mw=640), pentaerythrityl tetraoleate, neopentyl glycol diethylhexanoate, diethylhexyl sebacate and tricaprylate/tricaprate. The weight average molecular weight of these co-solvents is preferably from about 500 to about 1000, and more preferably from 500 to 800.

Examples of suitable high viscosity co-solvents which are compatible with the soft segment of the block copolymer include, but are not limited to, polyisobutene, hydrogenated polyisobutene, polybutene, hydrogenated polybutene, polydecene and hydrogenated polydecene. The weight average molecular weight of these co-solvents is preferably from about 2,500 to about 100,000, and more preferably from 3,000 to 10,000.

These co-solvents, if present, may preferably be employed in the composition of the invention in an amount of up to about 50% by weight; up to 40% by weight; up to 30% by weight; up to 25% by weight; all weights based on the weight of the composition.

Coloring Agents

According to the present invention, the compositions may optionally comprise at least one coloring agent (colorant). Suitable coloring agents include but are not limited to pigments, dyes, such as liposoluble dyes, nacreous pigments, and pearling agents. Typically, when the composition contains colorants, it is a make-up composition such as a mascara composition. Alternatively, when the composition does not contain colorants, it is a clear or transparent composition which can be used as a basecoat (or topcoat) prior to (or after) application of a make-up composition such as a mascara to eyelashes, or it can be used as a hair treatment composition such as, for example, a hair conditioner or mousse. However, it is possible that topcoats, basecoats, hair treatment products and the like could contain colorants, and/or that a mascara or make-up composition could contain little or no colorant.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow. The liposoluble dyes, when present, generally have a concentration ranging up to 20% by weight of the total weight of the composition, such as from 0.0001% to 6%.

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, be present in the composition in a concentration ranging up to 50% by weight of the total weight of the composition, such as from 0.1% to 20%, preferably from 0.1% to 15%.

The pigments, which may be used according to the present invention, may be chosen from white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present, the pigments may be present in the composition in a concentration ranging up to 50% by weight of the total weight of the composition, such as from 0.5% to 40%, and further such as from 2% to 30%. In the case of certain products, the pigments, including nacreous pigments, may, for example, represent up to 50% by weight of the composition.

According to particularly preferred embodiments, the compositions of the present invention are in the form of an emulsion. Suitable emulsion forms include but are not limited to oil-in-water, water-in-oil, oil-in-water-in-oil, water-in-oil-in-water and nanoemulsions (emulsions whose oil globules are of very fine particle size, that is to say that they have a number-average size of less than about 100 nanometers (nm)). Emulsions contain at least one oil phase and at least one aqueous phase. Typically speaking, emulsions contain surfactants or surfactant-like materials which provide stability to the emulsions and inhibit de-phasing of the emulsions.

In one embodiment, the compositions of the present invention are substantially free of silicone oils (i.e., contain less than about 1% of silicone oil). In another embodiment, the compositions are substantially free of non-silicone oils (i.e., contain less than about 1% of non-silicone oil). In another embodiment, the compositions are substantially free of non-volatile oils (i.e., contain less than about 1% of non-volatile oil). In yet another embodiment, the compositions are substantially free of volatile oils (i.e., contain less than about 1% of volatile oil).

Preferably, the oils, if present, represent from about 5% to about 80% by weight of the total weight of the composition, more preferably from about 10% to about 60% of the total weight of the composition, and most preferably from about 15% to about 50%, including all ranges and subranges therebetween.

Water, when present, preferably represents from about 1% to about 70% by weight of the total weight of the composition, more preferably from about 5% to about 60% of the total weight of the composition, and most preferably from about 10% to about 50%, including all ranges and subranges therebetween.

One particularly preferred embodiment of the present invention is a composition for application to keratin materials (hair or eyelashes) which is an emulsion but which is substantially free of TEA-stearate (that is, less than 0.25% of TEA-stearate) or free of TEA Stearate (that is, less than 0.05% TEA-stearate).

Additional Ingredients

The compositions of the present invention can also comprise any additive usually used in the field under consideration. For example, film forming agents, polyamide resins, dispersants, antioxidants, essential oils, preserving agents, fragrances, liposoluble polymers that are dispersible in the medium, fillers, neutralizing agents, cosmetic and dermatological active agents such as, for example, emollients, moisturizers, vitamins, anti-wrinkle agents, essential fatty acids, sunscreens, surfactants or emulsifiers, and mixtures thereof can be added. A non-exhaustive listing of such ingredients can be found in U.S. patent application Ser. No. 10/733,467, filed Dec. 12, 2003, the entire contents of which is hereby incorporated by reference. Further examples of suitable additional components can be found in the other references which have been incorporated by reference in this application, including but not limited to the applications from which this application claims priority. Still further examples of such additional ingredients may be found in the International Cosmetic Ingredient Dictionary and Handbook (9^{th} ed. 2002).

For example, at least one polyurethane/poly(meth)acrylate graft copolymer can optionally be added to the invention compositions. Suitable polyurethane/poly(meth)acrylate graft copolymers include but are not limited to those disclosed in U.S. patent application publication no. 2004-0136937, the entire contents of which is hereby incorporated by reference.

The polyurethane/poly(meth)acrylate graft copolymers may also be referred to as an interpenetrated polymer network (IPN) of a polyurethane and a poly(meth)acrylate. As used herein, the expression “interpenetrated polymer network” refers to a blend of two interlaced polymers, obtained by simultaneous polymerization and/or crosslinking of two types of monomer, the blend obtained having a single glass transition temperature.

Preferred IPNs include those which are commercially available from the company Air Products under the name Hybridur. An IPN that is particularly preferred is in the form of an aqueous dispersion of particles e.g., with a weight-average size of between 90 and 110 nm and a number-average size of about 80 nm. This IPN preferably has a glass transition temperature, Tg, ranging from about −60° C. to +100° C. An IPN of this type is available from Air Products under the trade name Hybridur 875 (INCI name: POLYURETHANE-2 (and) POLYMETHYL METHACRYLATE). Polyurethane/poly(meth)acrylics available from Air Products under the names Hybridur X-01602 and X 18693-21 are also preferred.

Preferred IPNs such as those discussed above are disclosed in U.S. patent application publication nos. 2003/0215476, 2004/0136937, 2005/0249763, the entire contents of all of which is hereby incorporated by reference.

According to preferred embodiments, the IPNs are polyurethane/poly(meth)acrylate graft copolymers having the following general structure:

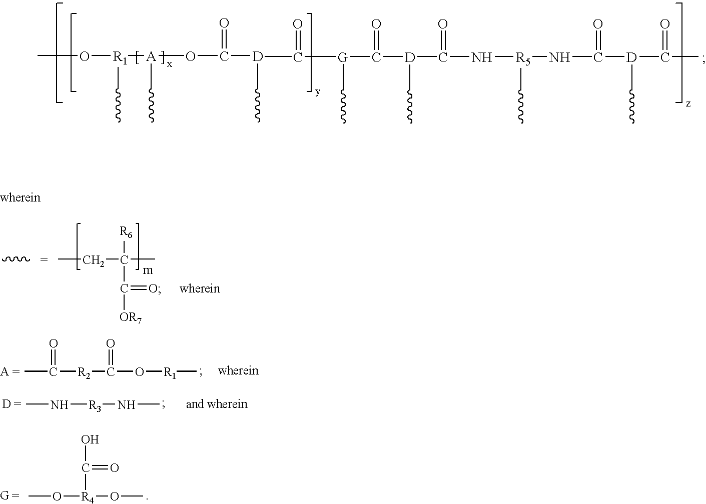

In the formula, R₁, R₂, R₃, R₄, R₅ and R₇ each independently represents an aliphatic hydrocarbon; m represents zero or a positive integer; R₆ represents hydrogen or methyl; and x, y and z each independently represents a positive integer. The graft copolymers may be provided in the form of aqueous dispersions. The graft copolymers may be added to the other components of the composition in powdered form as well.

Preferably, the polyurethane/poly(meth)acrylate graft copolymer is present in an amount sufficient to reduce tackiness of the composition. It is to be understood that the amount of polyurethane/poly(meth)acrylate graft copolymer needed to reduce tackiness of a composition should depend upon the amount of tacky ingredients (for example, tacky film forming agents) present in the composition. For example, the more tacky ingredients which are present, the more polyurethane/poly(meth)acrylate graft copolymer should be needed to reduce tackiness of the composition. In a particularly preferred embodiment, sufficient polyurethane/poly(meth)acrylate graft copolymer is present in the composition to substantially eliminate tackiness of the composition (that is, sufficient polyurethane/poly(meth)acrylate graft copolymer is present in the composition such that one or more characteristics associated with tackiness (for example, stickiness, clumping, poor spreadability, etc.) are not detectible upon application of the composition to a keratin material.

Generally speaking, preferred ranges of polyurethane/poly(meth)acrylate graft copolymer in the composition, when presente, are from about 0.1% to about 75% by weight of the total weight of the composition, more preferably from about 0.5% to about 70% of the total weight of the composition, more preferably from about 0.75% to about 50% of the total weight of the composition, more preferably from about 1% to about 30% of the total weight of the composition, and most preferably from about 1% to about 15%, including all ranges and subranges therebetween.

Also, modified clays can optionally be added to the invention compositions such as, for example, hectorites modified with an ammonium chloride of a C₁₀ to C₂₂ fatty acid, such as hectorite modified with distearyldimethylammonium chloride, also known as quatermium-18 bentonite, such as the products sold or made under the names Bentone 34 by the company Rheox, Claytone XL, Claytone 34 and Claytone 40 sold or made by the company Southern Clay, the modified clays known under the name quaternium-18 benzalkonium bentonites and sold or made under the names Claytone HT, Claytone GR and Claytone PS by the company Southern Clay, the clays modified with stearyldimethylbenzoylammonium chloride, known as steralkonium bentonites, such as the products sold or made under the names Claytone APA and Claytone AF by the company Southern Clay, and Baragel 24 sold or made by the company Rheox.

person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present).

Needless to say, the composition of the invention should be cosmetically or dermatologically acceptable, i.e., it should contain a non-toxic physiologically acceptable medium and should be able to be applied to the eyelashes of human beings.

According to preferred embodiments of the present invention, methods of increasing keratin material (hair or eyelash) volume and/or length comprising applying to keratin materials a keratin material volume- and/or length-increasing effective amount of a composition comprising at least one tackifier and at least one semi-crystalline alkyl(meth)acrylate are provided. The compositions may be applied to the keratin materials as needed, preferably once or twice daily, more preferably once daily and then preferably allowed to dry before subjecting to contact such as with clothing or other objects.

According to yet further embodiments of the present invention, methods of making-up keratin materials (hair or eyelashes) comprising applying a keratin material making-up effective amount of a composition comprising at least one tackifier and at least one semi-crystalline alkyl(meth)acrylate to keratin materials in need of such making-up are provided.

According to preferred embodiments of the present invention, methods of treating or caring for keratin materials (hair or eyelashes) by applying compositions of the present invention to the keratin materials in an amount sufficient to treat and/or care for the keratin materials are provided.

According to other preferred embodiments, methods of enhancing the appearance of keratin materials (hair or eyelashes) by applying compositions of the present invention to the keratin materials in an amount sufficient to enhance the appearance of the keratin materials are provided. In accordance with the preceding preferred embodiments, the compositions of the present invention are applied topically to keratin materials (hair or eyelashes) in an amount sufficient to treat, care for and/or make up the keratin materials, or to enhance the appearance of the keratin materials. The compositions may be applied to eyelashes as needed, preferably once or twice daily, more preferably once daily and then preferably allowed to dry before subjecting to contact such as with clothing or other objects.

According to yet other preferred embodiments, methods of supporting, strengthening, stiffening and/or reinforcing faux fibers by adding a supporting-, strengthening-, stiffening and/or reinforcing-effective amount of at least one semi-crystalline alkyl(meth)acrylate to a composition comprising at least one tackifier which, upon application to a keratin material, forms faux fibers on the keratin material are provided.

The present invention also envisages kits and/or prepackaged materials suitable for consumer use containing one or more compositions according to the description herein (for example, kits containing (1) a mascara; and (2) a basecoat and/or topcoat). The packaging and application device for any subject of the invention may be chosen and manufactured by persons skilled in the art on the basis of their general knowledge, and adapted according to the nature of the composition to be packaged. Indeed, the type of device to be used can be in particular linked to the consistency of the composition, in particular to its viscosity; it can also depend on the nature of the constituents present in the composition, such as the presence of volatile compounds.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLE 1

### Mascara

| Ingredient | Amount Present (% by weight) |
|---|---|
| Isododecane | 51 |
| Regalite 1100 | 19 |
| Kraton G 1657 M | 8 |
| Puresyn 150 | 0.8 |
| PolyStearyl Acrylate | 5 |
| Bentone | 4 |
| LAN Phase | 2 |
| Silicone Elastomer | 2 |
| Hybridur 875 | 3 |
| Syntran 5760 | 3 |
| Silica | 1 |
| Propylene Carbonate | 1.2 |

Composition of LAN Phase

| | |
|---|---|
| 0.7% | Phenoxyethanol |
| 0.3% | Disodium EDTA |
| 15.0% | Sodium Glycolate |
| 6.0% | Octylacrylamide/Acrylates/Butylaminoethylmethacrylate |
| | Copolymer |
| 0.5% | Tocopherol Acetate |
| 5.0% | Lecithin |
| 1.0% | Simethicone |
| 20.0% | Isoceteth-20 |
| 0.1% | Propylparaben |
| 0.1% | Methylparaben |
| 51.3% | Water |

### Procedure

1. In the main kettle, isododecane was heated to 65-75° C. Under mixing, Regalite 1100 was added and dissolved (about 15 minutes).
2. After the solid was completely dissolved, Kraton G 1657 M was added under vigorous mixing. The temperature was maintained between 65 and 75° C.
3. After all solids were dissolved, heating was stopped.
4. Puresyn 150 was added and mixing continued.
5. Polystearyl Acrylate and Bentone were added and mixed well until dissolved.
6. Silicone elastomer was added and mixed well. LAN Phase, Hybridur 875, Syntran 5760, and silica were added and mixed well.
7. When the temperature reached 55-60° C., propylene carbonate was added and mixed well.

What is claimed is:

1. A mascara comprising at least one coloring agent, at least one semi-crystalline alkyl(meth)acrylate, at least one tackifier and at least one block copolymer.

2. The mascara according to claim 1, wherein the at least one semi-crystalline alkyl(meth)acrylate is an alkyl acrylate.

3. The mascara according to claim 2, wherein the at least one semi-crystalline alkyl(meth)acrylate is a stearyl acrylate.

4. The mascara according to claim 1, wherein the at least one tackifier is a hydrogenated hydrocarbon resin.

5. The mascara according to claim 4, wherein the at least one tackifier is a hydrogenated styrene/methyl styrene/indene copolymer.

6. The mascara according to claim 3, wherein the at least one tackifier is a hydrogenated styrene/methyl styrene/indene copolymer.

7. The mascara according to claim 1, wherein the block copolymer is an A-B copolymer.

8. The mascara according to claim 1, wherein the block copolymer is an A-B-A copolymer.

9. The mascara according to claim 1, wherein the at least one semi-crystalline alkyl(meth)acrylate is present in an amount ranging from about 2% to about 25 % by weight with respect to the weight of the composition.

10. The mascara according to claim 1, wherein the at least one semi-crystalline alkyl(meth)acrylate is present in an amount ranging from about 4% to about 15% by weight with respect to the weight of the composition.

11. The mascara according to claim 1, wherein the mascara is anhydrous.

12. The mascara according to claim 1, wherein the mascara is in the form of an emulsion.

13. A method of making-up eyelashes comprising applying the mascara according to claim 1 to eyelashes.

14. A method of increasing eyelash volume or length comprising applying the mascara according to claim 1 to eyelashes.

15. A method of lengthening a keratin material comprising applying a composition comprising at least one semi-crystalline alkyl(meth)acrylate, at least one tackifier and at least one block copolymer to the keratin material in an amount sufficient to lengthen the keratin material.

16. The method of claim 15, wherein the keratin material is an eyelash.

17. A method of supporting, strengthening, stiffening and/or reinforcing faux fibers by adding a supporting-, strengthening-, stiffening- and/or reinforcing-effective amount of at least one semi-crystalline alkyl(meth)acrylate to a composition comprising at least one tackifier and at least one block copolymer which, upon application to a keratin material, forms faux fibers on the keratin material.
